(19) United States
(12) Patent Application Publication
(10) Pub. No.: US 2002/0102213 A1
(43) Pub. Date: Aug. 1, 2002

(54) Tissue mapping method and tissue map analyzer

(75) Inventors: Den?apos;etsu Sutoo, Tsukuba-shi (JP); Kayo Akiyama, Tsukuba-shi (JP); Kazunori Yabe, Makabe-gun (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: Yamato Scientific Co., Ltd., Tokyo (JP)

(21) Appl. No.: 10/057,967

(22) Filed: Jan. 29, 2002

(30) Foreign Application Priority Data
Jan. 30, 2001 (JP) 2001-021699

(51) Int. Cl. A61K 49/00; G06F 19/00; G01N 33/48; G01N 33/50; G06K 9/00

(52) U.S. Cl. 424/9.6; 702/19; 382/128

(57) ABSTRACT

A plurality of immunofluorescent reactions on the same brain specimen is analyzed and imaged correlatively at the cellular level. Information at a plurality of points on a tissue sample 9 is measured using a photometer 7 while two-dimensionally moving a scanning stage 5 on which the tissue sample 9 is placed. The measured signals are input to a memory 29 and stored therein, and a tissue map is created by obtaining two-dimensional information for the sample 9 from the memory 29. After reacting a reagent A′ specific to a substance A with the tissue sample to be mapped, a distribution image of the reaction areas of the reagent A′ is created by scanning the sample in the two-dimensional directions, and, after reacting a reagent B′ specific to a substance B with the same sample, a distribution image of the reaction areas of the reagent B′ is created by scanning the sample in the two-dimensional directions. These steps are repeatedly performed a necessary number of times, thereby creating distribution images of the reaction areas of different types of reagents on the same sample. A plurality of substances on the same sample 9 can be quantitatively analyzed from values such as the difference between or ratio of the distributions of these substances.

### BACKGROUND OF THE INVENTION

1. Field of the Invention

The present invention relates to a tissue mapping method and a tissue map analyzer. More particularly, the present invention relates to a tissue mapping method and a tissue map analyzer capable of simultaneously and correlatively analyzing and imaging a plurality of chemical reactions occurring on the same specimen at the cellular level.

2. Description of Background Art

Several tens of millions or more nerve cells are distributed in the human brain. Every emotion and behavior is controlled as a result of a chemical reaction occurring in each cell. Therefore, the major theme of brain research is to examine what kind of chemical reaction occurs in which region of the brain at the cellular level.

Conventionally, the distributions of a plurality of substances in the same brain specimen have been examined using the brains of animals as experimental materials employing a histochemical method which includes labeling the substances with antibodies labeled with different fluorescent substances (for example, an antibody labeled with fluoresceine isothiocyanate (FITC) for substance A, and an antibody labeled with rhodamine for substance B), and photographing the specimen.

Conventionally, a biochemical method has also been employed. In the biochemical method, samples are collected by cutting the specimen into sections using a knife or the like, or punching out only thin sections using a pipe, and the resulting samples are biochemically analyzed.

A problem in brain research is to simultaneously and correlatively analyze a plurality of chemical reactions occurring on the same specimen at the cellular level. Solving this problem is expected to be a very effective means to analyze various types of nervous diseases and to develop prevention and treatment methods.

The histochemical method enables the distributions of the substances to be observed visually by indicating the substance A by green and the substance B by red on the photograph, for example. However, the substances cannot be determined quantitatively by this method. A method of separating fluorescence originating from each fluorescent substance in the same specimen has been studied by replacing a filter in a microscope. However, it is impossible to completely separate the fluorescence using the filter, thereby giving rise to problems relating to quantitation.

In the biochemical method, the operations of collecting the samples by cutting the specimen into sections are complicated and inefficient. Moreover, it is impossible to collect samples from ultra-microareas. Furthermore, complicated operations before analysis increase errors when analyzing unstable chemical substances.

Therefore, neither the histochemical method nor the biochemical method can enable a plurality of chemical reactions on the same specimen to be analyzed correlatively at the cellular level.

The tissue samples collected from patients who have contracted rare diseases are valuable and the amount of samples is limited. Therefore, it is desired that the distributions of different types of substances be measured using the same sample. These samples are stored for a long period of time. When new findings are obtained relating to the disease, it is necessary to study the disease using the stored samples. Specifically, a method of quantitatively evaluating the distributions of a plurality of substances using the same specimen is demanded.

The present invention has been achieved to solve the above-described problems. An object of the present invention is to provide a tissue mapping method and a tissue map analyzer capable of correlatively analyzing the distributions of a plurality of substances or chemical reactions on the same specimen at the cellular level and imaging the analytical results, by immunofluorescently staining a tissue sample such as a brain specimen with a reagent specific to the substance to be measured such as antibodies, and optically analyzing the distributions of the intensities of fluorescent substances on the specimen.

### SUMMARY OF THE INVENTION

One aspect of the present invention provides a tissue mapping method comprising placing a tissue sample on a stage, measuring information at a plurality of points on the sample using a measuring device while moving the stage two-dimensionally, inputting signals measured by the measuring device to a memory and storing the signals therein, and creating a tissue map by obtaining two-dimensional information about the sample from the memory,

wherein, after reacting a reagent A′ specific to a substance A with the tissue sample to be mapped, a distribution image of the reaction areas of the reagent A′ is created by scanning the sample in the two-dimensional directions, and subsequently, after reacting a reagent B′ specific to a substance B with the same sample, a distribution image of the reaction areas of the reagent B′ is created by scanning the sample in the two-dimensional directions, wherein these steps are repeatedly performed a necessary number of times, thereby creating distribution images of the reaction areas of different types of reagents on the same sample.

Therefore, the tissue sample such as a brain specimen is specifically stained with the reagent A′. This sample is moved in the two-dimensional directions by the stage, whereby the distribution image of the photometric signal intensities of the reagent A′ is created based on optical characteristics (reflected light, transmitted light, fluorescence, or the like) of the reagent A′ distributed on the sample. The same sample is then specifically stained with the reagent B′. The distribution image of the photometric signal intensities of the reagent B′ distributed on the same sample is created through the same procedures. The distributions of the photometric signal intensities of the reagents A′ and B′ respectively correspond to the distributions of the substances A and B present in the brain specimen or the like. The distributions of a plurality of chemical substances on the same sample are calculated into values such as the difference between or ratio of the signal values at the same position and imaged at the cellular level. This enables quantitative analysis of substances, whereby a plurality of chemical reactions occurring in the brain can be analyzed correlatively at the cellular level.

As the reagent A′ specific to the substance A or the reagent B′ specific to the substance B, antibodies or antibodies labeled with a fluorescent substance are preferable. The brain specimen is immunofluorescently stained by staining the sample using such antibodies as primary antibodies or secondary antibodies. This sample is moved by the stage in the two-dimensional directions, whereby the distribution image of the intensities of the fluorescent substance with which the antibody specifically combining with the substance A distributed on the sample is labeled, is created. The same sample is then immunofluorescently stained with an antibody or antibody labeled with a fluorescent substance specific to the substance B. The distribution image of the intensities of the fluorescent substance with which the antibody specifically combining with the substance B distributed on the same sample is labeled, is created through the same procedures. The distributions of a plurality of chemical substances on the same sample are calculated into values such as the difference between or ratio of the signal intensities of the fluorescence intensities at the same position and imaged at the cellular level. This enables quantitative analysis of substances, whereby a plurality of chemical reactions occurring in the brain can be correlatively analyzed at the cellular level.

Another aspect of the present invention provides a tissue map analyzer comprising:

a measuring device for measuring information at one point on a tissue sample,

a stage which moves two-dimensionally with the sample being placed thereon,

a memory for storing information concerning coordinates and signal intensities at a plurality of points on the sample to which measured signals from the measuring device are input while moving the stage,

a plurality of divisional memories provided in the memory, each of which stores data on distribution of the reaction areas of each reagent reacted with the same tissue sample obtained by the measuring device by scanning the sample in the two-dimensional directions,

a processor for calculating analytical values of the data stored in two different divisional memories, and

an image processor for creating a distribution image of each reagent based on the data stored in each divisional memory in the memory, and creating a distribution image based on the analytical values calculated by the processor.

Therefore, the tissue sample such as a brain specimen is specifically stained with a reagent A′ specific to a substance A. This sample is moved in the two-dimensional directions by the stage, whereby the distribution image of the photometric signal intensities of the reagent A′ distributed on the sample is created. The same sample is specifically stained with a reagent B′ specific to a substance B. The distribution image of the photometric signal intensities of the reagent B′ distributed on the same sample is created through the same procedures. The distributions of a plurality of chemical substances on the same sample are calculated into values such as the difference between or ratio of the signal intensities of the fluorescence intensities at the same position by the processor and imaged at the cellular level. This enables easy, reliable quantitative analysis of the substances.

In this tissue map analyzer, a plurality of divisional memories may comprise:

a first memory for storing data on the distribution of the reaction areas of a reagent A′ specific to a substance A obtained by the measuring device by scanning the tissue sample with which the reagent A′ is reacted in the two-dimensional directions,

a second memory for storing data on the distribution of the reaction areas of a reagent B′ specific to a substance B obtained by the measuring device by scanning the same tissue sample with which the reagent B′ is reacted in the two-dimensional directions, and

a third memory for storing data on the distribution of the reaction areas of a reagent C′ specific to a substance C obtained by the measuring device by scanning the same tissue sample with which the reagent C′ is reacted in the two-dimensional directions.

Therefore, the data on the distribution of the reaction areas of the reagent A′ on the tissue sample with which the reagent A′ is reacted is stored in the first memory, the data relating to the reagent B′ is stored in the second memory, and the data relating to the reagent C′ is stored in the third memory. Because of this, image processing of the reaction areas corresponding to the reagents A′, B′, and C′, and calculation of values such as the difference between or ratio of the data on the reaction areas corresponding to different types of reagents become easy.

This tissue map analyzer may comprise a sample positioning means for positioning the same tissue sample at a specific position of the stage.

Therefore, the reaction areas of a plurality of reagents on the same tissue sample can be imaged while being overlapped precisely, whereby the values such as the difference between or ratio of the data stored in two different divisional memories can be imaged reliably.

### BRIEF DESCRIPTION OF THE DRAWINGS

FIG. 1 is an oblique view showing a tissue map analyzer according to an embodiment of the present invention.

FIG. 2 is a structural block diagram showing the tissue map analyzer according to the embodiment of the present invention.

FIG. 3 is an oblique view showing part of a scanning stage according to the embodiment of the present invention.

FIG. 4 is an oblique view showing part of another scanning stage according to the embodiment of the present invention.

FIG. 5 is a view showing examples of an optical system of the tissue map analyzer according to the embodiment of the present invention.

FIG. 6 shows maps indicating the distributions of each substance in the brain tissue of a rat, in which a symbol A shows the distribution of tyrosine hydroxylase (TH), a symbol B shows the distribution of calmodulin (CaM), and a symbol C shows the distribution of calmodulin-dependent protein kinase II (CaMKII).

FIG. 7 shows maps indicating the results calculated based on data on the distributions of CaM and CaMKII shown in FIG. 6, in which a symbol A shows areas in which the amount of CaMKII is greater than that of CaM obtained by subtracting the data on CaM (B in FIG. 6) from the data on CaMKII (C in FIG. 6), a symbol B shows areas in which the amount of CaM is greater than that of CaMKII obtained by subtracting the data on CaMKII (C in FIG. 6) from the data on CaM (B in FIG. 6), and a symbol C shows areas in which the amount of CaMKII is relatively greater than that of CaM obtained by calculating the ratio of CaMKII to CaM.

### DETAILED DESCRIPTION OF THE PREFERRED EMBODIMENT

An embodiment of the present invention is described below in detail with reference to the drawings.

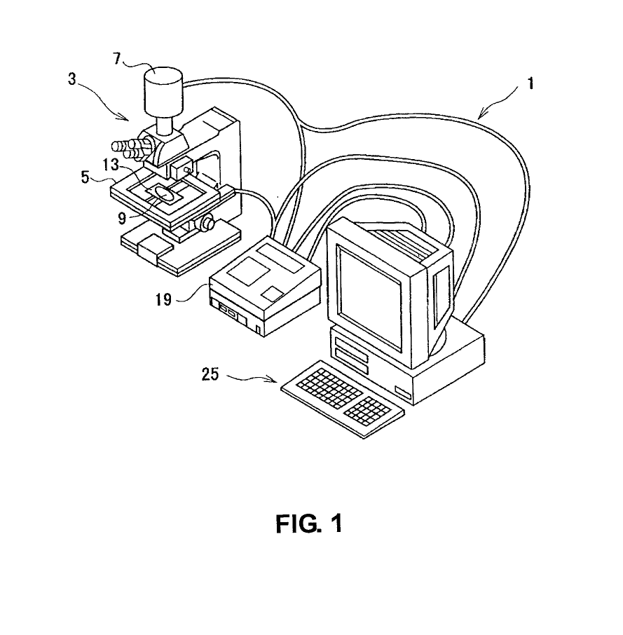

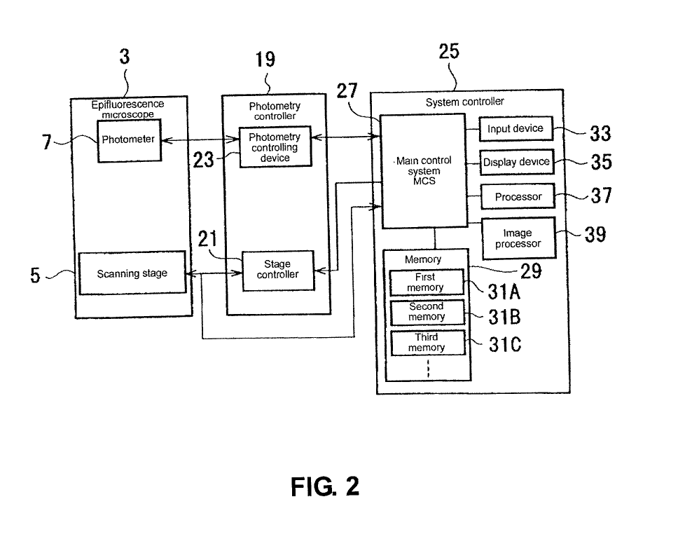

Referring to FIGS. 1 and 2, a tissue map analyzer 1 according to the present embodiment determines the two-dimensional distributions of photometry data of a sample using an epifluorescence microscope 3. The microscope 3 includes an autoscanning stage (hereinafter called “scanning stage”) 5, and a measurement device such as a photometer 7.

The photometer 7 includes a photomultiplier, which is the most reliable detector in view of sensitivity and quantitativity. An interference filter and a photometry diaphragm are inserted in front of the detector. An excitation beam can optionally be narrowed by a field diaphragm and an aperture diaphragm (minimum spot diameter at the surface of specimen 9:0.8 μmφ when using 100-power objective lens, 6 μmφ when using 20-power objective lens). Therefore, only light from a microarea of the target sample such as the specimen 9 can be measured selectively.

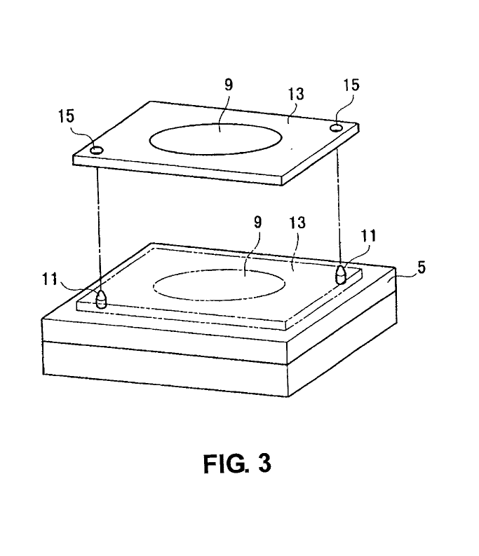

Referring to FIG. 3, at least two positioning pins 11 are provided on the scanning stage 5 as sample positioning means for installing the same specimen 9 as the same tissue sample at a specific position of the scanning stage 5. The same number of positioning holes 15 as the positioning pins 11 are formed in a glass slide 13 to which the specimen 9 is attached. The positioning pins 11 can be inserted into or removed from the positioning holes 15.

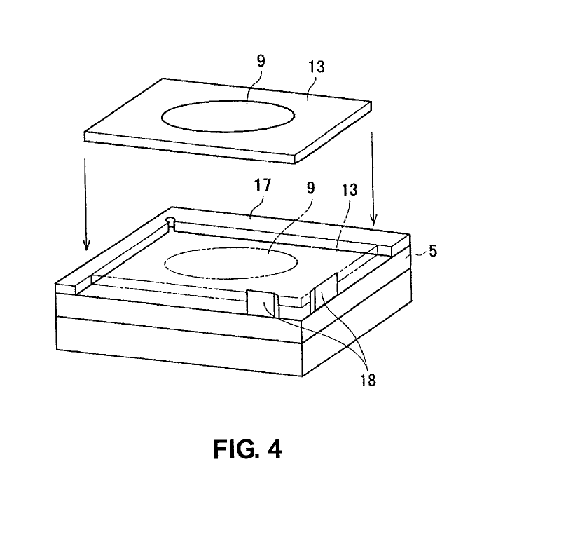

As another example of the sample positioning means, a support member 17 to which two adjacent sides of the glass slide 13 are attached is provided on the scanning stage 5, as shown in FIG. 4. The glass slide is secured at a specific position using slide holders 18 provided on the remaining two sides. The support member 17 may comprise at least three support pins. There are no specific limitations to the sample positioning means insofar as the glass slide 13 can be secured at a specific position of the scanning stage 5 and removed therefrom. Positioning markers may be provided on the scanning stage 5. The glass slide 13 may be positioned using these markers each time.

A photometry controller 19 includes a stage controller 21 for controlling the movement of the scanning stage 5 in the XY directions, and a photometry controlling device 23 for controlling the photometer 7.

A system controller 25 includes a main control system MCS 27 for outputting a command signal to the stage controller 21 and the photometry controlling device 23, and a memory 29 for storing the photometry data.

A plurality of divisional memories 31 is provided in the memory 29. After the same specimen 9 as the same tissue sample to be measured is reacted with each of the reagents A′, B′, C′ . . . , each of the divisional memories 31 stores data on the distribution of each reagent (data on the intensities of transmitted light, reflected light, fluorescent light, or the like) measured by the photometer 7 by scanning the specimen 9 using the scanning stage 5 two-dimensionally in the XY directions.

A plurality of divisional memories 31 is provided for each of the reagents A′, B′, C′ . . . , such as a first memory 31A which stores data on the distribution of the reaction areas of the reagent A′ obtained by the photometer 7 by scanning the tissue specimen 9, with which the reagent A′ is reacted, in the XY directions, a second memory 31B which stores data on the distribution of the reaction areas of the reagent B′ obtained by the photometer 7 by scanning the same tissue specimen 9, with which the reagent B′ is reacted, in the XY directions, and a third memory 31C which stores data on the distribution of the reaction areas of the reagent C′ obtained by the photometer 7 by scanning the same tissue specimen 9, with which the reagent C′ is reacted, in the XY directions.

The system controller 25 includes an input device 33 for inputting data or a command, such as a keyboard, and a display device 35, such as a CRT. The system controller 25 includes a processor 37 for calculating the analytical values such as the difference between or ratio of the data stored in two different divisional memories 31 among the data stored in a plurality of divisional memories 31A, 31B, 31C . . . . The system controller 25 further includes an image processor 39 for creating distribution images (tissue maps) of each of the reagents A′, B′, C′ . . . based on the data stored in each of the divisional memories 31A, 31B, 31C, . . . in the memory 29. The image processor 39 may create the distribution images (tissue maps) based on the analytical values calculated by the processor 37.

The scanning stage 5 scans freely at a step width of 0.25 μm or more in the X or Y direction on a command from the photometry controller 19. The scanning speed is 100 photometry-points/sec, and the scanning range can optionally be selected within 140×140 mm. The intensity of transmitted light, reflected light, or fluorescence is measured at each photometry point while moving the specimen 9 at a constant speed. The measured results are sent to the system controller 25. Since each point is irradiated with the excitation beam for only 0.01 sec, even in the case of using fluorescence, fading of fluorescence due to irradiation is scarcely observed.

In more detail, light emitted from the specimen 9 placed on the scanning stage 5 is detected by the photometer 7 through the microscope 3. The detected photometry signals are output to the main control system MCS 27 of the system controller 25 through the photometry controlling device 23 and stored in the memory 29. The system controller 25 and the photometry controlling device 23 are connected through a photometry data transfer line.

The main control system MCS 27 outputs a command signal to the stage controller 21, thereby causing the scanning stage 5 to be moved to an optional measuring point. Pulse signals output to the scanning stage 5 from the stage controller 21 are also output to the main control system MCS 27. The main control system MCS 27 repeatedly stores the data based on the pulse signals from the stage controller 21, whereby the two-dimensional photometric distribution of the specimen 9 is obtained.

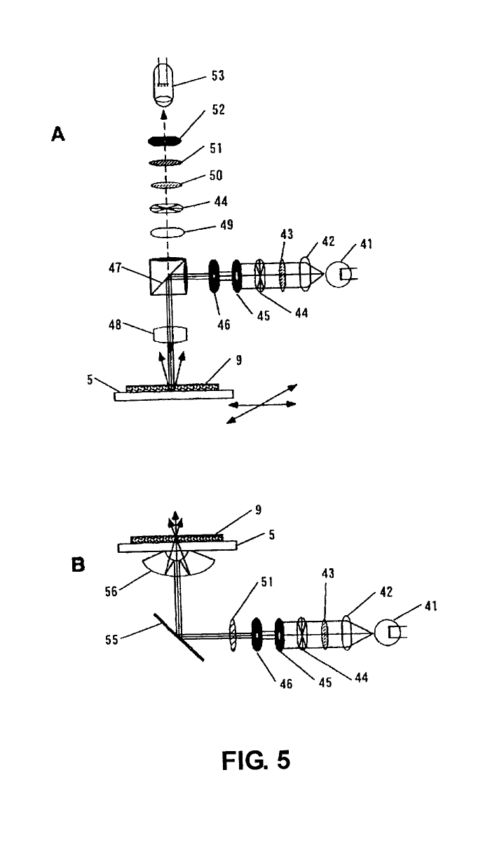

The tissue map analyzer of the present invention measures reflected light or fluorescence from the sample, or light transmitted through the sample. Examples of an optical system used in the present invention are shown in FIG. 5. A in FIG. 5 illustrates an optical system in the case of measuring reflected light or fluorescence. B in FIG. 5 illustrates an optical system in the case of measuring transmitted light.

In A in FIG. 5, light emitted from a halogen lamp 41 is narrowed through a collector lens 42, heat insulating filter 43, shutter 44, aperture diaphragm 45, and field diaphragm 46, reflected by a dichroic mirror 47, and concentrated on the specimen (tissue sample) 9 through an objective lens 48. Light reflected by the sample or fluorescence excited by the irradiated light passes into the dichroic mirror 47 through the objective lens 48, and enters a measuring system. In the measuring system, the light or fluorescence enters a photomultiplier 53 through a projective lens 49, shutter 44, infrared cut filter 50, band-pass filter (interference filter) 51, and photometry diaphragm 52, whereby the intensity of the light or fluorescence is measured. A symbol 5 indicates the scanning stage. The scanning stage is moved in the X or Y direction, whereby the surface of the sample is scanned.

B in FIG. 5 illustrates the case of measuring transmitted light. Light emitted from the halogen lamp 41 is narrowed through the collector lens 42, heat insulating filter 43, shutter 44, aperture diaphragm 45, and field diaphragm 46, reflected by a mirror 55 through the band-pass filter (interference filter) 51, and irradiated to the specimen (tissue sample) 9. Light transmitted through the sample is guided to a measuring system (not shown) similar to that shown in A, whereby the intensity of the light is measured. A symbol 56 indicates a condenser for concentrating the light on the measurement area on the specimen 9.

The photometry operations are summarized briefly below taking the case of using fluorescence as an example.

(1) The microarea (photometry point) of the specimen 9 which is immunofluorescently stained is irradiated with a narrowed excitation beam.

(2) Fluorescence emitted from this microarea is concentrated on the photomultiplier 53 through the objective lens 48 and the photometry diaphragm 52, and the intensity of the fluorescence is measured therein.

(3) The specimen 9 is moved to the next photometry point by the scanning stage 5, and the intensity of fluorescence is measured.

(4) The data on the fluorescence intensities in each microarea is stored in the memory 29 of the system controller 25. The image of the entire scanned area is constructed and displayed in the display device 35 as a colored or monochromatic image.

A tissue mapping method of the embodiment of the present invention is described below.

The mapping method of the present invention causes a specific reagent to combine with a substance present in the tissue sample, and measures optical changes or characteristics occurring in the combined area using transmitted light, reflected light, fluorescence, or the like (photometry). Therefore, in the case where the reagent to be combined has optical characteristics in which the reagent emits fluorescence or has a specific absorption wavelength, photometry can be performed using these characteristics as optical markers. Even if the reagent itself has no optical marker, in the case where the optical characteristics of the reagent such as the absorption wavelength change when combined with a specific substance, photometry can be performed using these optical characteristics as the optical markers.

The reagent specific to the substance used in the present invention means a substance which specifically combines or reacts with this substance. The substance which specifically combines or reacts with a specific substance (specific reagent) is selected depending on the type of substance to be measured. For example, an antibody to the substance, a ligand which combines with a specific receptor, a substrate for a specific enzyme, and the like can be given. Depending on the substance to be measured, a substance which is known to combine with a specific substance such as the biotin-avidin system may be applied.

A fluorescent substance may be combined with an antibody or ligand so that the antibody or ligand emits fluorescence. Photometry may be performed using the emitted fluorescence as an optical marker. In the case of using an antibody, the tissue sample may be immunofluorescently stained using a double-antibody method in which an antibody specific to the substance to be measured is combined with the substance, and stained using an antibody to this antibody with which a fluorescent substance is combined (fluorescence-labeled anti-antibody).

The antibody to the substance to be measured is obtained by immunizing animals such as mice, rabbits, sheep, or goats according to a conventional method. Depending on the substance to be measured, commercially available products of antibodies may be used.

Fluorescence-labeled anti-antibodies are commercially available, such as an anti-rabbit IgG antibody (serum), anti-sheep IgG antibody (serum), and anti-mouse IgG antibody (serum) which are labeled with a fluorescent substance. These products may be used.

Immunohistochemical staining can be performed by using an antibody to the substance to be measured with which a fluorescent substance is combined according to a conventional method. However, since the anti-antibodies labeled with a fluorescent substance are commercially available as described above, immunohistochemical staining by the double-antibody method using these fluorescence-labeled anti-antibodies (serum) is preferable because of ease of use and general convenience.

As the fluorescent substances used for labeling, fluorescein isothiocyanate (FITC, excitation wavelength; 496 nm, fluorescence wavelength; 518 nm), tetramethylol isothiocyanate (TRITC, excitation wavelength; 554 nm, fluorescence wavelength; 565 nm), rhodamines such as lissamine rhodamine sulfonyl chloride (LRSC, excitation wavelength; 572 nm, fluorescence wavelength; 590 nm) and sulforhodamine 101 acid chloride (Texas Red, excitation wavelength; 592 nm, fluorescence wavelength; 610 nm), and the like can be given. It is preferable to use the same fluorescent substance in a series of mappings from the viewpoint of quantitativity. However, different fluorescent substances may be used by correcting the relation between the fluorescence intensity emitted from these materials and the concentrations.

The tissue specimen is prepared by treating the tissue sample placed on a glass slide using a reagent which specifically combines with the substance to be measured, and removing the uncombined reagent by rinsing. In the case of using the double-antibody method, the specimen is then treated with an anti-antibody with which a fluorescent substance is combined, and the uncombined fluorescence-labeled anti-antibody is removed by rinsing to obtain a tissue specimen in which the substance to be measured is labeled with the fluorescent substance. The tissue specimen thus prepared is placed on the scanning stage and scanned, whereby the intensity of light at each point is measured.

In the case where the tissue specimen emits fluorescence such as the case where the tissue specimen is stained with a pigment which emits fluorescence (such as eosine or methyl green), or the tissue itself emits fluorescence, the specimen before the antibody is combined may be photometered in advance, and the state when the antibody is combined may be determined based on the measured values.

In the case where a substance which specifically combines with the substance to be measured is known, such as in the case of a receptor and ligand or an enzyme and substrate, the ligand or substrate or a derivative of the ligand or substrate with which a fluorescent substance is combined may be used as the specific reagent. In the case where the substance to be measured is a protein, such as an enzyme, and an antibody to the protein can be produced, the antibody can be easily obtained by immunizing using this protein as an antigen. This antibody may be used as the specific reagent.

In the case where the substance to be measured is a low-molecular-weight substance, such as amino acid, peptide, or biogenic amine which cannot produce the antibody, these substances can be mapped by performing immunofluorescent staining as follows (Geffard, M. et al., Brain Res. 294: 161-165; 1984 and Geffard, M. et al., J. Neurochem. 42: 1593-1599; 1984).

The following description is given taking the case of mapping a biogenic amine in the brain of a mouse as an example. A biogenic amine is conjugated to albumin by glutaraldehyde in vitro to obtain a high-molecular-weight amine-albumin conjugated product. A rabbit is immunized using the conjugated product as an antigen to obtain an antibody from the serum.

When a mouse is perfused through the heart with glutaraldehyde, the same conjugated reaction occurs in the living body. A biogenic amine and albumin are conjugated by the perfused glutaraldehyde in the brain of the mouse, whereby an amine-albumin conjugated product is produced and fixed at a position at which the biogenic amine was present. The brain is then sliced and mounted on a glass slide to obtain a specimen. The specimen is treated with an anti-amine-albumin conjugated product antibody of a rabbit prepared separately, whereby an anti-biogenic amine-albumin conjugated product rabbit antibody is combined with the mouse biogenic amine-albumin conjugated product. The resulting product is treated with a fluorescence-labeled anti-rabbit IgG antibody (serum) to prepare a specimen.

As described above, in the present invention, not only a protein present in the brain, but also a low-molecular-weight substance such as biogenic amine can be immunofluorescently stained, whereby these substances can be mapped by measuring the fluorescence using the tissue map analyzer of the present invention.

### EXAMPLES

A method of multi-mapping a plurality of substances in the same specimen of the brain of a rat using the map analyzer is described below taking tyrosine hydroxylase, calmodulin, and calmodulin-dependent protein kinase II as examples.

Immunofluorescent staining was performed by a double antibody method using an anti-tyrosine hydroxylase antibody (TH antibody, reagent A′), an anti-calmodulin antibody (CaM antibody, reagent B′), and an anti-calmodulin-dependent protein kinase II antibody (CaMKII antibody, reagent C′) as specific reagents and using anti-IgG antibodies (serum) to these antibodies labeled with a fluorescent substance (FITC) as secondary antibodies.

The TH antibody was purchased from Chemicon International, Inc. (Catalog No. AB151). This antibody is produced from a rabbit using TH purified from a rat adrenal medullary tumor tissue as an antigen.

The CaM antibody was purchased from Transformation Research, Inc. (Catalog No. 5016). This antibody is produced from a sheep using CaM purified from a bovine testes.

The CaMKII antibody was purchased from Chemicon International, Inc. (Catalog No. MAB8699). This antibody is produced by immunizing a mouse with purified CaMKII, forming a 6G9 hybridoma clone by fusing lymphocytes in the spleen with a mouse myeloma cell (NS1/SP2), producing an antibody in the ascites, and purifying the antibody by protein A fractionation.

The FITC-labeled anti-rabbit IgG goat serum was purchased from Zymed Lab., Inc. (Catalog No. 65-6111).

The FITC-labeled anti-sheep IgG rabbit serum was purchased from Vector Lab., Inc. (Catalog No. F1-6000).

The FITC-labeled anti-mouse IgG goat serum was purchased from American Qualex International, Inc. (Catalog No. A106FU).

A tissue specimen was prepared as follows.

A rat was anesthetized with pentobarbital and perfused through the heart with a mixed solution of 4% paraformaldehyde and 0.2% glutaraldehyde. The brain of the rat was removed, frozen with liquid nitrogen or dry ice, sliced to a thickness of 20 μm using a cryostat, and attached to a glass slide coated with gelatin to prepare a specimen.

This rat brain specimen was then immunofluorescently stained using the above antibodies. TH was immunofluorescently stained by the double-antibody method using FITC. The operations were as follows.

(1) The above specimen was rinsed with PBS (phosphate-buffered saline) for 20 minutes.

(2) The specimen was pretreated with normal goat serum at 4° C. for two hours, whereby the non-specific binding of the anti-rabbit IgG goat serum was blocked.

(3) The specimen was rinsed with PBS for 60 minutes.

(4) A 200-fold dilution of the TH antibody was placed on the specimen and reacted at 4° C. for 12 hours.

(5) The specimen was rinsed with PBS for 20 minutes three times.

(6) A 100-fold dilution of the FITC-labeled anti-rabbit IgG goat serum was placed on the specimen and reacted at room temperature for three hours.

(7) The specimen was rinsed with PBS for 20 minutes three times.

(8) The specimen was embedded in 10% glycerine PBS and immediately measured using the tissue map analyzer.

The same specimen for which the TH distribution was measured was immunohistochemically stained using a 200-fold dilution of the CaM antibody and a 200-fold dilution of the FITC-labeled anti-sheep IgG rabbit serum in the same manner as in the case of using the TH antibody. It was then measured using the tissue map analyzer. Glycerine on the specimen was removed by rinsing with PBS before staining.

The immunohistochemical fluorescence intensity of CaM was determined from the difference in fluorescence intensity before and after the staining of CaM in each microarea of the brain specimen.

The same specimen for which the CaM distribution was measured was immunofluorescently stained using a 100-fold dilution of the CaMKII antibody and a 40-fold dilution of the FITC-labeled anti-mouse IgG goat serum in the same manner as in the case of using the CaM antibody. It was then measured using the tissue map analyzer.

The immunohistochemical fluorescence intensity of CaMKII was determined from the difference in fluorescence intensity before and after the staining of CaMKII in each microarea of the brain specimen.

A method of analyzing the distributions (mapping) of each substance of the same specimen thus obtained is described below.

The entire area of the specimen was measured at intervals of 20 μum to obtain about 400,000 pieces of data from each specimen. The distribution of the immunohistochemical fluorescence intensities of the substance first stained (TH) was directly obtained by measuring the stained specimen. The background of the specimen may be measured before staining, and the distribution of the immunohistochemical fluorescence intensities of TH may be determined by subtracting these values. The distributions of the immunohistochemical fluorescence intensities of the substances stained after the first substance (CaM and CaMKII) were determined from the difference in fluorescence intensity before and after staining of each substance.

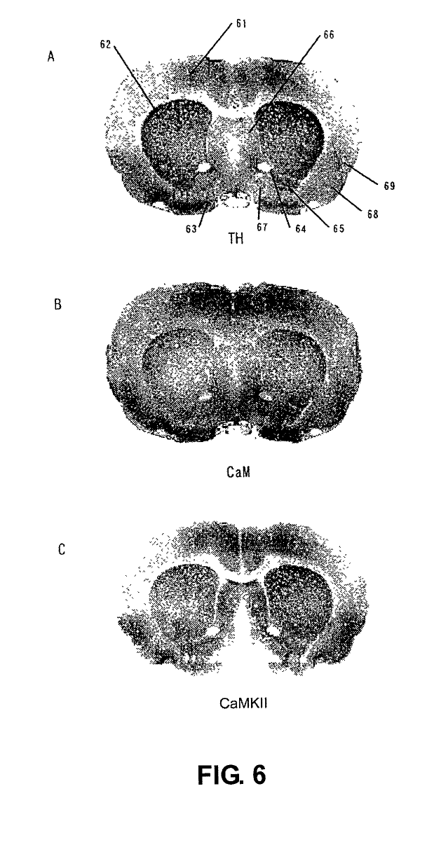

Specifically, the brain specimen is stained with the TH antibody, and the fluorescence intensities in the entire area of the specimen are measured. This data (first data) shows the distribution of TH. An image shown in FIG. 6A is obtained by stratifying the resulting fluorescence intensities at 8-256 levels and displaying the fluorescence intensity at each point of the specimen using the difference in brightness (black and white) or colors. In this image (black and white in the drawing), the amount of TH is greater in the blacker areas. Specifically, TH is localized in the blacker areas. The brain specimen was prepared by slicing the brain of the rat in the coronal plane. The upper part of the image corresponds to the parietal region of the rat. In the figure, 61 indicates the motor cortex, 62 indicates the striatum, 63 indicates the olfactory tubercle, 64 indicates the anterior commissure, 65 indicates the nucleus accumbens, 66 indicates the lateral septal nucleus, 67 indicates the diagonal band, 68 indicates the piriform cortex, and 69 indicates the insular cortex. As is clear from the resulting image, TH is localized in the striatum 62, nucleus accumbens 65, olfactory tubercle 63, and motor cortex 61.

The resulting data (first data) is stored in the first memory 31A in the memory 29 of the system controller 25.

The same specimen is then stained with the CaM antibody. The stained specimen is placed at the same position on the scanning stage and measured under the same conditions. This fluorescence intensity data (second data) is sent to the system controller 25, and stored in the second memory 31B in the memory 29 of the system controller 25. The data indicating the distribution of CaM is obtained by subtracting the first value from the second value at each photometry point by the processor 37. The image shown in FIG. 6B is obtained by displaying the stratified data.

The same specimen is then stained with the CaMKII antibody and measured in the same manner. The fluorescence intensity data (third data) is sent to the system controller 25, and stored in the third memory 31C in the memory 29 of the system controller 25. The data indicating the distribution of CaMKII is obtained by subtracting the second value from the third value at each photometry point by the processor 37. The image shown in FIG. 6C is obtained by displaying the stratified data.

The quantitative images of TH, CaM, and CaMKII are obtained in this manner using the tissue map analyzer. The quantitative images shown in FIGS. 6A to 6C and the raw data are stored in the memory 29.

Comparison of the distributions of each substance obtained from the same specimen is described below.

Since the quantitative images of TH, CaM, and CaMKII can be stored in the memory 29, immunohistochemical fluorescence intensities between the substances in each microarea can be compared and analyzed quantitatively using the tissue map analyzer.

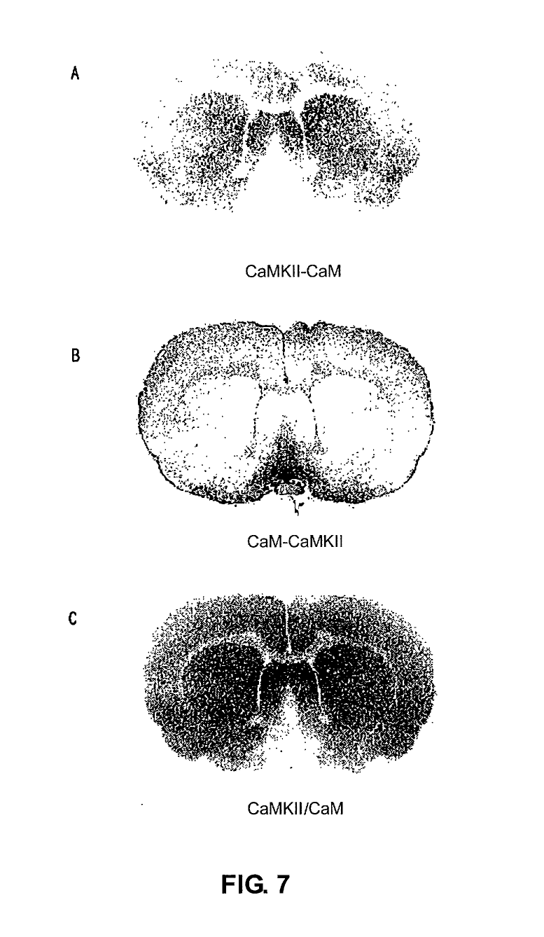

For example, an image shown in FIG. 7A is obtained by subtracting the immunohistochemical fluorescence intensity of CaM (FIG. 6B) from the pure immunohistochemical fluorescence intensity of CaMKII (FIG. 6C) in each microarea of the specimen, and imaging the differences. In the image shown in FIG. 7A, the black areas indicate areas in which the amount of CaMKII is greater than that of CaM.

On the contrary, an image shown in FIG. 7B is obtained by subtracting the fluorescence intensities of CaMKII (FIG. 6C) from the fluorescence intensities of CaM (FIG. 6B) and imaging the differences. The black areas indicate areas in which the amount of CaM is greater than that of CaMKII.

An image shown in FIG. 7C is obtained by dividing the fluorescence intensities of CaMKII (FIG. 6C) by the fluorescence intensities of CaM (FIG. 6B) and imaging these ratios (CaMKII/CaM). The black areas indicate areas in which the amount of CaMKII is relatively greater than that of CaM.

From the images thus obtained, the amount of TH, CaM, and CaMKII is greater in the lateral area of the striatum 62, nucleus accumbens 65, olfactory tubercle 63, and motor cortex 61. Therefore, these areas are considered to play an important role in the brain. The amount of CaMKII is greater in the lateral septal nucleus 66, medial area of the striatum 62, piriform cortex 68, and insular cortex 69 in comparison with CaM. On the contrary, the amount of CaM is greater in the diagonal band 67 in comparison with CaMKII.

According to the tissue map analyzer of the present invention, since the excitation beam is narrowed by the field diaphragm and the aperture diaphragm, and each photometry point is irradiated with the excitation beam for only 0.01 sec., fading of fluorescence is scarcely observed by only one occurrence of photometry. In the experiments, the same data was obtained even after repeating the analysis fifty times. Therefore, even if the operations consisting of staining, photometry, staining, and photometry are repeatedly performed as in the example, values with no errors can be obtained. Moreover, the tissue map analyzer, which uses the photomultiplier as the detector, excels in quantitativity by two digits or more in comparison with an image analyzer using a TV camera.

The present invention is not limited to the above-described embodiment. Various modifications and variations are possible without departing from the spirit and scope of the present invention.

In the present embodiment, the photometry controller 19 and the system controller 25 are separately provided. However, the photometry controller 19 may be included in the system controller 25. The main control system MCS 27 and the memory 29 may be included in the photometry controller 19.

According to the tissue mapping method of the present invention, the tissue sample such as a brain specimen is specifically stained with the specific reagent A′ (immunofluorescently stained in the case of using an antibody as specific reagent A′). This sample is moved in the two-dimensional directions by the scanning stage and measured using the tissue map analyzer, whereby the distribution of the reagent A′ can be imaged based on optical characteristics (such as fluorescence intensities) of the reagent A′ distributed on the sample. The same sample is immunofluorescently stained with the specific reagent B′. The distribution of the intensities of the fluorescent substance in the reagent B′ distributed on the same sample can be imaged by photometry through the same procedures. Therefore, the distribution of a plurality of chemical reactions on the same sample can be calculated through values, such as the difference between or ratio of these values, and imaged at the cellular level. This enables easy, reliable quantitative analysis of the substances. Moreover, since the method of the present invention is nondestructive and is not affected by the conditions of the sample before measurement, the distributions of different substances on the same specimen can be measured repeatedly.

According to the tissue map analyzer of the present invention, a tissue sample such as a brain specimen is specifically stained with a specific reagent A′ (immunofluorescently stained in the case of using an antibody as specific reagent A′). This sample is moved in the two-dimensional directions by the scanning stage and measured using the tissue map analyzer, whereby the distribution of the reagent A′ can be imaged based on optical characteristics (such as fluorescence intensities) of the reagent A′ distributed on the sample. The same sample is stained with the specific reagent B′ in the same manner. The distribution of the reagent B′ distributed on the same sample can be imaged by photometry through the same procedure. Therefore, the distribution of a plurality of substances on the same sample can be calculated through values, such as the difference between or ratio of the values, and imaged correlatively at the cellular level. This enables easy, reliable quantitative analysis of the substances.

According to the present invention, the data on the distribution of the reaction areas of the specific reagent A′ on the tissue sample with which the reagent A′ is reacted is stored in the first memory, the data relating to the specific reagent B′ is stored in the second memory, and the data relating to the specific reagent C′ is stored in the third memory. Because of this, image processing of the reaction areas corresponding to the reagents A′, B′, and C′, and calculation of the analytical values, such as the difference between or ratio of the data of the reaction areas corresponding to different reagents, become easy.

Moreover, according to the present invention, since the position of the same tissue sample which is reacted with a plurality of reagents can be overlapped reliably, the measurement operations become easy and accuracy of the resulting images can be improved.

Obviously, numerous modifications and variations of the present invention are possible in light of the above teachings. It is therefore to be understood that, within the scope of the appended claims, the invention may be practiced other than as specifically described herein.

What is claimed is:

1. A tissue mapping method comprising placing a tissue sample on a stage, measuring information at a plurality of points on the sample using a measuring device while moving the stage two-dimensionally, inputting signals measured by the measuring device to a memory and storing the signals therein, and creating a tissue map by obtaining two-dimensional information of the sample from the memory,
wherein, after reacting a reagent A′ specific to a substance A with the tissue sample to be mapped, a distribution image of the reaction areas of the reagent A′ is created by scanning the sample in the two-dimensional directions, and subsequently, after reacting a reagent B′ specific to a substance B with the same sample, a distribution image of the reaction areas of the reagent B′ is created by scanning the sample in the two-dimensional directions, wherein these steps are repeatedly performed a necessary number of times, thereby creating distribution images of the reaction areas of different types of reagents on the same sample.

2. The tissue mapping method according to claim 1, wherein the reagent is an antibody or an antibody labeled with a fluorescent substance.

3. A tissue map analyzer comprising:
a measuring device for measuring information at one point on a tissue sample,
a stage which moves two-dimensionally with the sample being placed thereon,
a memory for storing information concerning coordinates and signal intensities at a plurality of points on the sample to which measured signals from the measuring device are input while moving the stage,
a plurality of divisional memories provided in the memory, each of which stores data on distribution of the reaction areas of each reagent reacted with the same tissue sample obtained by scanning the sample in the two-dimensional directions,
a processor for calculating analytical values of the data stored in two different divisional memories, and
an image processor for creating a distribution image of each reagent based on the data stored in each divisional memory in the memory and creating a distribution image based on the analytical values calculated by the processor.

4. The tissue map analyzer according to claim 3,
wherein a plurality of divisional memories comprises:
a first memory for storing data on the distribution of the reaction areas of a reagent A′ specific to a substance A obtained by the measuring device by scanning the tissue sample with which the reagent A′ is reacted in the two-dimensional directions,
a second memory for storing data on the distribution of the reaction areas of a reagent B′ specific to a substance B obtained by the measuring device by scanning the same tissue sample with which the reagent B′ is reacted in the two-dimensional directions, and
a third memory for storing data on the distribution of the reaction areas of a reagent C′ specific to a substance C obtained by the measuring device by scanning the same tissue sample with which the reagent C′ is reacted in the two-dimensional directions.

5. The tissue map analyzer according to claim 3 or 4, further comprising:
a sample positioning means for positioning the same tissue sample at a specific position of the stage.
